(19) United States
(12) Patent Application Publication
(10) Pub. No.: US 2026/0200965 A1
(43) Pub. Date: Jul. 16, 2026

(54) NOVEL COMPOUND DERIVED FROM LORANTHUS TANAKAE AND ANTICANCER USE THEREOF

(71) Applicant: KOREA INSTITUTE OF ORIENTAL MEDICINE, Daejeon (KR)

(72) Inventors: A Yeong LEE, Daejeon (KR); Jun LEE, Gyeongsangnam-do (KR); Mee Hyun LEE, Jeollanam-do (KR); Byeong Cheol MOON, Jeollanam-do (KR); Hyo Seon KIM, Seoul (KR); Young Hye SEO, Jeollanam-do (KR); Seung Mok RYU, Jeollanam-do (KR); Hyeon Hwa NAM, Jeollanam-do (KR)

(73) Assignee: KOREA INSTITUTE OF ORIENTAL MEDICINE, Daejeon (KR)

(21) Appl. No.: 19/139,278

(22) PCT Filed: Dec. 14, 2023
(86) PCT No.: PCT/KR2023/020684

§ 371 Date: Jun. 13, 2025

(30) Foreign Application Priority Data
Dec. 14, 2022 (KR) 10-2022-0174442

(51) Int. Cl. C07H 17/07; A61K 9/00; A61K 31/7048; A61K 36/185; A61P 35/00; C07H 1/08

(52) U.S. Cl. CPC C07H 17/07; A61K 9/0056; A61K 31/7048; A61K 36/185; A61P 35/00; C07H 1/08; A61K 2236/333; A61K 2236/39; A61K 2236/53

(57) ABSTRACT

The present invention relates to a novel compound derived from Loranthus tanakae and an anticancer use thereof. More specifically, the present invention provides: lotanoside B (indicated as rhamnetin-3-O-Rhamnoside-4′-O-(6″-O-cinnamoyl)-glucoside(Rhamnetin-3-O-rhamnoside-4′-O-(6″-O-cinnamoyl)-glucoside according to the IUPAC nomenclature) that is represented by formula 1 below; an isomer or pharmaceutically acceptable salt thereof; a pharmaceutical composition and functional health food composition containing same; and a method for separating lotanoside B represented by formula 1 below and/or rhamnetin-3-O-glucoside represented by formula 2 below.

### TECHNICAL FIELD

The present invention relates to a novel compound derived from Loranthus tanakae and anticancer use thereof. More specifically, the present invention provides: Lotanoside B (indicated as rhamnetin-3-O-rhamnoside-4′-O-(6″-O-cinnamoyl)-glucoside according to the IUPAC nomenclature) that is represented by Chemical Formula 1 below; an isomer or pharmaceutically acceptable salt thereof; a pharmaceutical composition and functional health food composition including the same; and a method for isolating Lotanoside B represented by Chemical Formula 1 below and/or rhamnetin-3-O-glucoside represented by Chemical Formula 2 below.

### BACKGROUND ART

Common cancer treatments include surgery, chemotherapy and radiotherapy. Surgery is classified into diagnostic surgery, radical surgery, preventive surgery and palliative surgery depending on the purpose. Diagnostic surgery is performed to determine the classification and type of tumor, and radical surgery is a method to treat early-stage cancer by removing both the lymph nodes surrounding the tumor and the primary lesion. Preventive surgery is a method to help the prevention of cancer by removing some polyps, which are known as precancerous lesions, in advance, and palliative surgery is a method to relieve cancer symptoms by reducing the size of the tumor.

The growth and differentiation of normal cells in our body are strictly regulated, but cancer cells show abnormal patterns in cell growth control and continuously proliferate. Therefore, suppressing cancer cell proliferation is important, and chemotherapy is one of them. It is used for the purpose of treating cancer by destroying cancer cells and preventing recurrence, and in cancer that cannot be cured, it is used to prolong life by suppressing the growth or spread of cancer cells. Applicable drugs include alkylating drugs, antimetabolites and the like. Alkylating drugs directly bind to DNA, destroying the DNA molecule itself and damaging the single-strand or double-strand structure, thereby inhibiting the growth and division of cancer cells. Representative drugs include platinum compounds such as Cisplatin and Carboplatin, and nitrogen mustard drugs such as Mechlorethamine (nitrogen mustard) and Cyclophosphamide. Since antimetabolites have a similar structure to the metabolites required for DNA replication in normal cells, they competitively bind to the biosynthetic enzymes of purines and pyrimidines with normal metabolites, thereby inhibiting their action so as to exhibit an anticancer effect. Representative examples include pyrimidine derivatives such as fluorouracil (5-FU), capecitabine and cytarabine, and purine derivatives such as mercaptopurine (6-MP). Radiation therapy is a method of destroying cancer cells using radiation, and it is sometimes used to alleviate the patient's symptoms when treatment is no longer possible.

However, these cancer treatment methods effectively suppress the growth of cancer, but also cause side effects. In the case of surgery, side effects such as pulmonary complications, anastomotic leakage (the part where the intestine is cut and connected), intestinal obstruction, urinary problems and sexual dysfunction may occur. In the case of anticancer chemotherapy, the toxicity of the drug spreads not only to cancer cells but also to normal cells, and thus, some complications may occur. In addition, there are digestive disorders and enteritis, leukocyte or thrombocytopenia, immunosuppression, skin pruritus and alopecia, neurotoxicity, kidney and liver toxicity. In radiation therapy, since some radiation is administered to normal tissue as well as cancer tissue, complications such as poor wound healing, skin necrosis, bone marrow suppression, proctitis and bleeding may occur. Therefore, interest in extracts of natural materials with fewer side effects and anticancer effects mentioned above is increasing, and the development of safe and effective natural medicines is required.

Meanwhile, mistletoe is a semi-parasitic plant that can photosynthesize but absorbs water and nutrients from the host tree. It has been used as a natural medicine to treat hyperlipidemia, cancer and diabetes in Europe, and the herbal medicine ‘Sanggi-saeng ( [symbol] )’ that strengthens the liver, kidneys and muscles and bones in oriental medicine also belongs thereto. There are three types of mistletoe that are native to Korea: Viscum album var. coloratum, Korthalsella japonica and Loranthus yadoriki, which grow in Gangwon-do, Gyeongsang-do and Jeju-do, and the relatively rare type of mistletoe, Loranthus tanakae, which grows parasitically on deciduous broadleaf trees in the mountains of Gangwon-do, Gyeongsang-do and Jeju-do.

The mistletoe is a deciduous small shrub belonging to the Loranthaceae family, and it is named after the type of mistletoe whose inflorescences and fruits hang long like tails. It differs from other mistletoes in that its fruits are relatively smaller and darker yellow, hang in long rows and are deciduous, meaning that there are no leaves in winter.

With regard to the physiological activity of Loranthus tanakae Franch. & Sav, the antioxidant or whitening effects of kaempferol 3-rhamnoside, rhamnetin 3-rhamnoside and rhamnocitrin 3-rhamnoside isolated from Loranthus tanakae Franch. & Sav are disclosed in Patent Document 1, and the antidiabetic effect of an extract of Loranthus tanakae Franch. & Sav is disclosed in Patent Document 2.

### RELATED ART DOCUMENTS

### Patent Documents

(Patent Document 1) Korean Patent Laid-Open Publication No. 10-2018-0001325
(Patent Document 2) Korean Patent Laid-Open Publication No. 10-2019-0044237

### DISCLOSURE

### Technical Problem

Under these circumstances, the inventors of the present invention have confirmed that a novel compound isolated from Loranthus tanakae Franch. & Sav has an effect of inhibiting or delaying tumor development by inhibiting cell transformation by epidermal growth factor (EGF) or tumor promoting factor phorbol ester 12-O-tetradecanoylphorbol-13-acetate (TPA), and that it can be safely applied to the human body because it does not show toxicity even at high concentrations, thereby completing the present invention.

Therefore, an object of the present invention is to provide a novel compound isolated from Loranthus tanakae Franch. & Sav and an anticancer composition using the same.

With regard to the use, another object of the present invention is to provide a method for preventing, ameliorating or treating cancer by using a novel compound isolated from Loranthus tanakae Franch. & Sav.

With regard to the use, still another object of the present invention is to provide the use of a novel compound isolated from Loranthus tanakae Franch. & Sav in the manufacture of a medicament or health functional food for preventing, ameliorating or treating cancer.

Another object of the present invention is to provide an anticancer composition using an extract of Loranthus tanakae Franch. & Sav or a fraction thereof.

With regard to the use, still another object of the present invention is to provide a method for preventing, ameliorating or treating cancer by using an extract of Loranthus tanakae Franch. & Sav or a fraction thereof.

With regard to the use, still another object of the present invention is to provide the use of an extract of Loranthus tanakae Franch. & Sav or a fraction thereof in the manufacture of a medicament or health functional food for preventing, ameliorating or treating cancer.

Still another object of the present invention is to provide a method for isolating a compound exhibiting anticancer activity from Loranthus tanakae Franch. & Sav.

### Technical Solution

In order to solve the above-described problems, the present invention provides Lotanoside B represented by Chemical Formula 1, an isomer thereof or a pharmaceutically acceptable salt thereof:

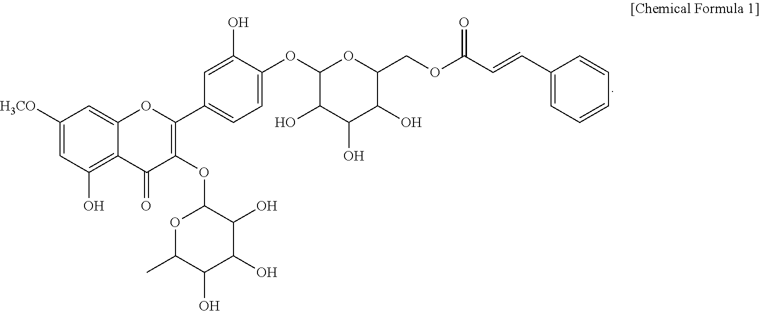 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1]

In addition, the isomer may include a racemate, an enantiomer, a diastereomer, a mixture of enantiomers or a mixture of diastereomers.

In addition, the Lotanoside B may be isolated from Loranthus tanakae Franch. & Sav.

In addition, the Lotanoside B may be isolated from a whole plant of Loranthus tanakae Franch. & Sav.

In addition, the Lotanoside B, isomer thereof or pharmaceutically acceptable salt thereof may have an effect of inhibiting cancer cells or inhibiting or delaying tumor development.

Additionally, the present invention provides a pharmaceutical composition, including the above-described compound, an isomer thereof or a pharmaceutically acceptable salt thereof as an active ingredient; and a health functional food composition including the above-described compound, an isomer thereof or a sitologically acceptable salt thereof as an active ingredient.

In addition, the Lotanoside B, isomer thereof or pharmaceutically acceptable salt thereof may be for preventing, ameliorating or treating cancer.

In addition, the pharmaceutical composition may be used as an anticancer adjuvant.

With regard to the use, the present invention further provides a method for preventing, ameliorating or treating cancer, including administering Lotanoside B (rhamnetin-3-O-rhamnoside-4′-O-(6″-O-cinnamoyl)-glucoside) represented by Chemical Formula 1 above, an isomer thereof or a pharmaceutically or acceptable salt thereof to a subject in need thereof

With regard to the use, the present invention provides the use of Lotanoside B (rhamnetin-3-O-rhamnoside-4′-O-(6″-O-cinnamoyl)-glucoside) represented by Chemical Formula 1 above, an isomer thereof or a pharmaceutically or sitologically acceptable salt thereof for the manufacture of a medicament or health functional food for preventing, ameliorating or treating cancer.

Furthermore, the present invention provides a health functional food composition for preventing or ameliorating cancer, including an extract of Loranthus tanakae Franch. & Sav including Lotanoside B represented by Chemical Formula 1 aboveor a fraction thereof as an active ingredient.

With regard to the use, the present invention further provides a method for preventing, ameliorating or treating cancer, including administering an extract of Loranthus tanakae Franch. & Sav including Lotanoside B represented by Chemical Formula 1 above or a fraction thereof to a subject in need thereof.

With regard to the use, the present invention further provides the use of an extract of Loranthus tanakae Franch. & Sav including Lotanoside B represented by Chemical Formula 1 above or a fraction thereof in the manufacture of a medicament or health functional food for preventing, ameliorating or treating cancer.

In addition, the extract of Loranthus tanakae Franch. & Sav may be a C₁ to C₄ lower alcohol, water or mixed solvent extract thereof.

In addition, the extract of Loranthus tanakae Franch. & Sav may be an ethyl acetate fraction of a C₁ to C₄ lower alcohol, water or mixed solvent extract thereof.

In addition, the extract of Loranthus tanakae Franch. & Sav or a fraction thereof may further include rhamnetin-3-O-glucoside represented by Chemical Formula 2 below:

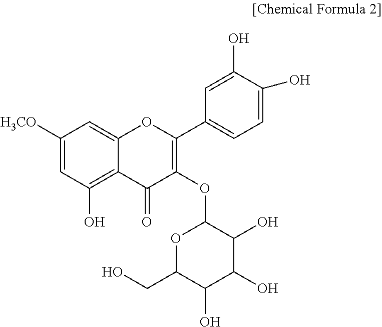 [SMILES: COc1cc(O)c2c(=O)c(OC3OC(CO)C(O)C(O)C3O)c(-c3ccc(O)c(O)c3)oc2c1]

In addition, the pharmaceutical composition may be used as an anticancer adjuvant.

Additionally, the present invention provides a method for isolating Lotanoside B represented by Chemical Formula 1 above and/or rhamnetin-3-O-glucoside represented by Chemical Formula 2 above from Loranthus tanakae Franch. & Sav, including:
(a) extracting Loranthus tanakae Franch. & Sav with an ethanol solvent to obtain a C₁ to C₄ lower alcohol, water or mixed solvent extract thereof;
(b) sequentially fractionating the extract with n-hexane, methylene chloride, ethyl acetate and n-butanol in this order to obtain an ethyl acetate fraction;
(c) performing flash chromatography on the ethyl acetate fraction using a water-methanol mixture solution as an extract solvent to obtain a fraction;
(d) performing reverse-phase flash chromatography on the fraction obtained after performing flash chromatography using a methanol solvent as an extract solvent to obtain a fraction; and
(e) performing reverse-phase flash chromatography and then performing normal-phase flash chromatography on the fraction obtained using a chloroform-methanol-water mixture solution as an extraction solvent to purify Lotanoside B represented by Chemical Formula 1 above and/or rhamnetin-3-O-glucoside represented by Chemical Formula 2 above.

In addition, the Loranthus tanakae Franch. & Sav may use a whole plant.

In addition, the mixed solvent of a C₁ to C₄ lower alcohol and water in step (a) may be a 30 to 80% ethanol solvent.

In addition, the water-methanol mixture solution of step (c) may be applied with a concentration gradient from 100% water to 100% methanol.

In addition, the methanol solvent of step (d) may be applied with a concentration gradient from 50% methanol to 100% methanol.

In addition, the chloroform-methanol-water mixture solution of step (e) may be applied at a concentration gradient of 7 to 9:1 to 3:0.1 to 2.

### Advantageous Effects

The Lotanoside B provided in the present invention exhibits excellent anticancer activity at low concentrations compared to kaempferol, which is known to have anticancer activity, and unlike the existing anticancer agent 5-FU, it does not exhibit toxicity even at high concentrations, and thus can be safely applied to the human body. Accordingly, it can be effectively used for the prevention, amelioration or treatment of cancer, and can prevent side effects caused by excessive use. In addition, the extract of Loranthus tanakae Franch. & Sav or a fraction thereof provided in the present invention includes not only Lotanoside B but also rhamnetin-3-O-glucoside, which has been confirmed to have individual anticancer activity. Therefore, the extract of Loranthus tanakae Franch. & Sav including at least one of the above compounds or a fraction thereof also has high utility as an anticancer agent or anticancer adjuvant.

### DESCRIPTION OF DRAWINGS

FIG. 1 shows the results of observing the growth inhibition effect of EGF or TPA-induced cell transformation colony of an extract of Loranthus tanakae Franch. & Sav.

FIG. 2 shows the HPLC pattern analysis results of a 70% ethanol extract of Loranthus tanakae Franch. & Sav, Lotanoside B and rhamnetin-3-O-glucoside in order.

FIG. 3 shows the HRMS analysis results of Lotanoside B.

FIG. 4 shows the ¹H NMR spectrum (600 MHz, methanol-d4) of Lotanoside B.

FIG. 5 shows the ¹³C NMR spectrum (150 MHz, methanol-d4) of Lotanoside B.

FIG. 6 shows the COSY-NMR results of Lotanoside B.

FIG. 7 shows the HMBC-NMR results of Lotanoside B.

FIG. 8 shows the HSQC-NMR results of Lotanoside B.

FIG. 9 shows the ¹H NMR spectrum (600 MHz, methanol-d4) of rhamnetin-3-O-glucoside.

FIG. 10 shows the ¹³C NMR spectrum (150 MHz, methanol-d4) of rhamnetin-3-O-glucoside.

FIG. 11 shows the results of evaluating the cytotoxicity of Lotanoside B in normal human skin fibroblasts.

FIG. 12 shows the results of evaluating the EGF-induced cell transformation colony growth inhibition effect of Lotanoside B.

FIG. 13 shows the results of evaluating the TPA-induced cell transformation colony growth inhibition effect of Lotanoside B.

FIG. 14 shows the results of evaluating the melanoma cancer cell growth inhibition effect of Lotanoside B using the WST-8 assay.

FIG. 15 shows the results of evaluating the inhibitory effect of rhamnetin-3-O-glucoside on the growth of EGF-induced cell transformation colonies.

FIG. 16 shows the results of evaluating the inhibitory effect of rhamnetin-3-O-glucoside on the growth of TPA-induced cell transformation colonies.

FIGS. 17A to 17D show the results of confirming the skin cancer prevention effect of Lotanoside B in a skin cancer-induced animal model. FIG. 17A shows the mouse body weight of each group in Table 1, FIG. 17B shows the number of tumors of each group in Table 1, FIG. 17C shows the tumor incidence rate of each group in Table 1, and FIG. 17D shows the tumor area of each group in Table 1.

### BEST MODE

Hereinafter, the present invention will be described in more detail.

Meanwhile, each description and exemplary embodiment disclosed in the present invention can also be applied to each other description and embodiment. That is, all combinations of various elements disclosed in the present invention fall within the scope of the present invention. In addition, the scope of the present invention cannot be said to be limited by the specific description described below.

In addition, a person having ordinary skill in the art can recognize or confirm many equivalents to the specific embodiments of the present invention described in the present application using only routine experiments. In addition, such equivalents are intended to be included in the present invention.

As described above, the inventors of the present invention isolated a novel compound from Loranthus tanakae Franch. & Sav and confirmed its excellent cancer cell inhibition effect, tumor development inhibition effect or tumor development delay effect.

Therefore, a first aspect of the present invention relates to a novel compound, which is Lotanoside B, an isomer or a pharmaceutically acceptable salt thereof, represented by Chemical Formula 1 below:

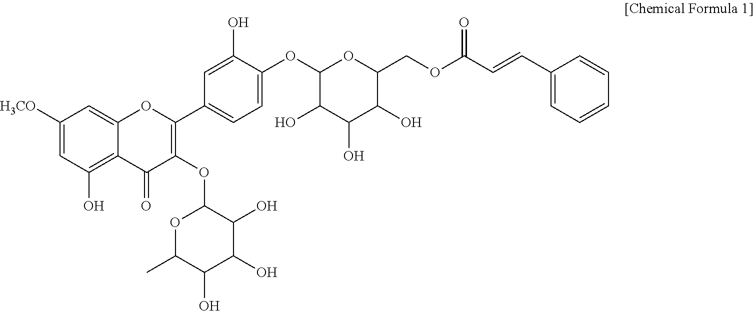 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1]

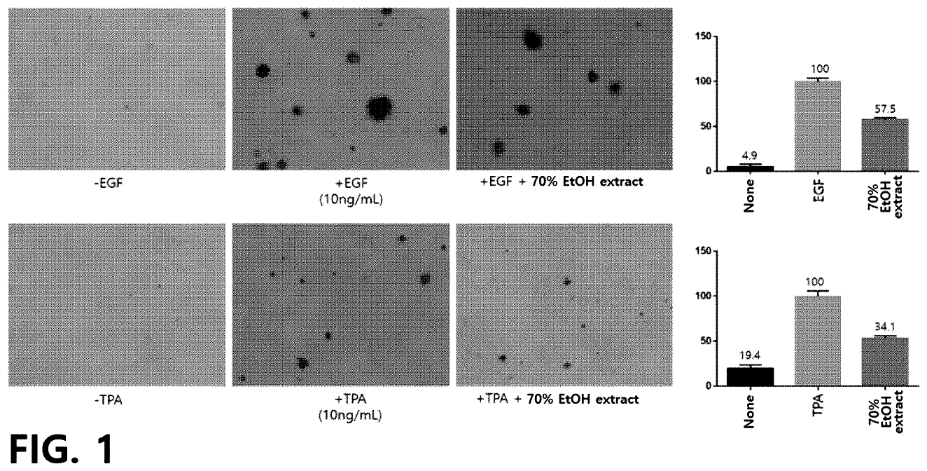

In a specific embodiment of the present invention, the anticancer activity of an extract of Loranthus tanakae Franch. & Sav was evaluated in an EGF-induced carcinogenesis model and a TPA-induced carcinogenesis model. As a result, as confirmed in FIG. 1, the extract of Loranthus tanakae Franch. & Sav showed an inhibition rate of 42.5% compared to the EGF treatment group in the EGF-induced carcinogenesis model, and an inhibition rate of 65.9% compared to the TPA treatment group in the TPA-induced carcinogenesis model.

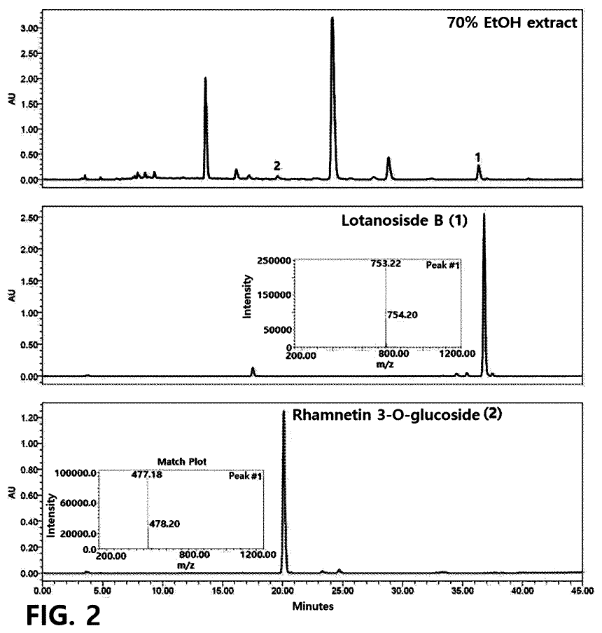

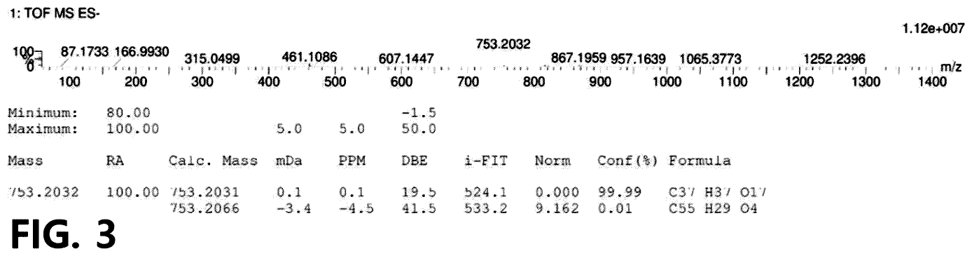

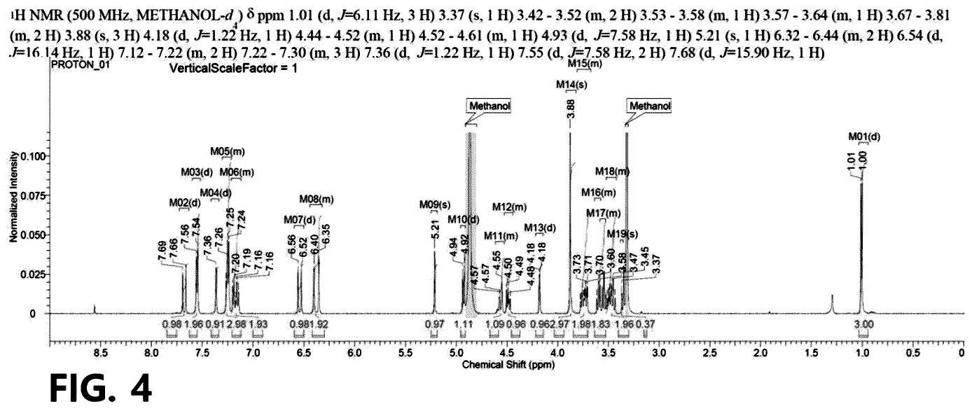

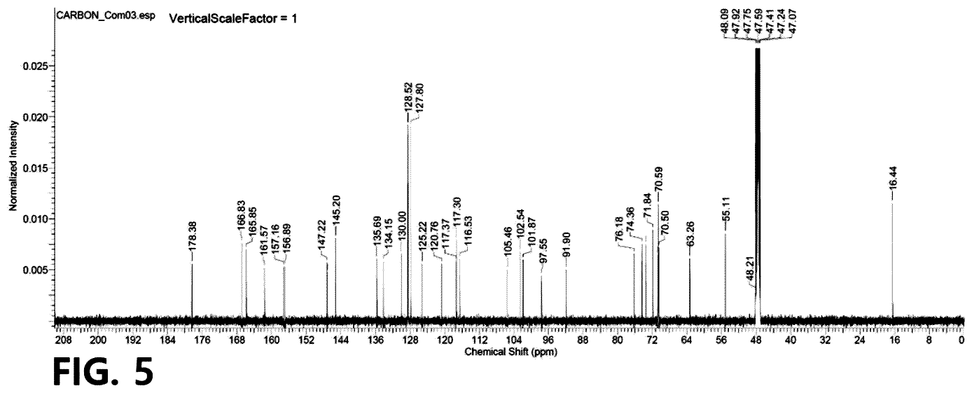

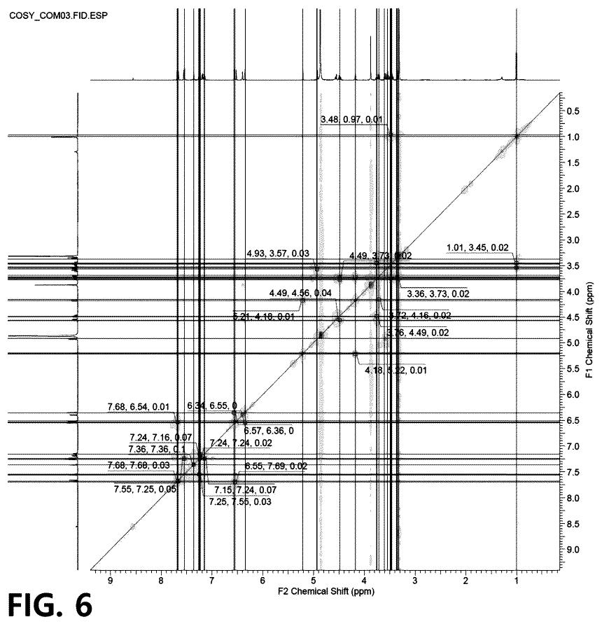

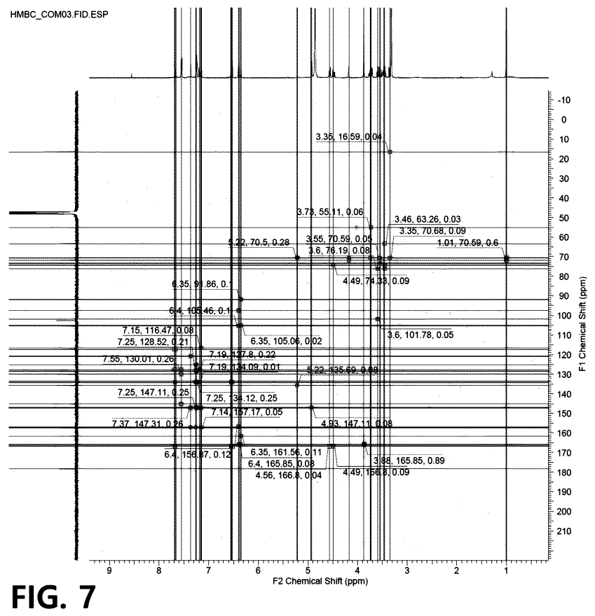

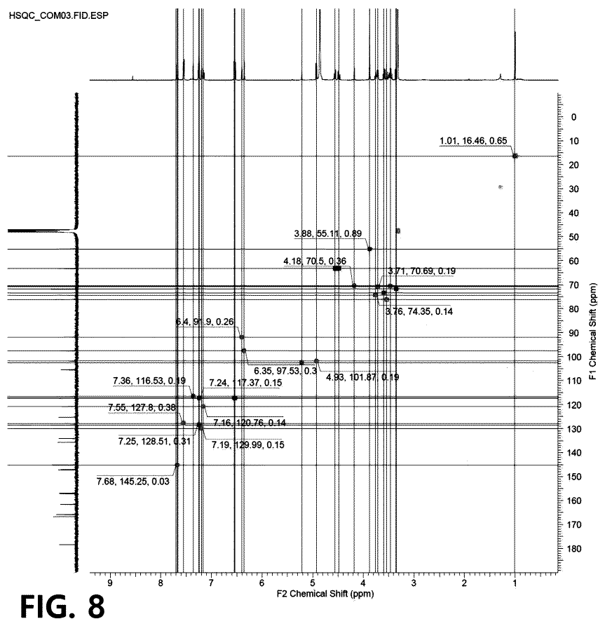

Accordingly, the inventors of the present invention identified individual components included in the extract of Loranthus tanakae Franch. & Sav through HPLC pattern analysis of the extract of Loranthus tanakae Franch. & Sav, as shown in FIG. 2. The compound structure of each component indicated as 1 in the HPLC pattern of the 70% ethanol extract of FIG. 2 was confirmed through HRMS, 1D-NMR or 2D-NMR analysis. As a result, as shown in FIGS. 3 to 8, the compound of the component indicated as 1 was confirmed to be a compound designated as rhamnetin-3-O-rhamnoside-4′-O-(6″-O-cinnamoyl)-glucoside according to the IUPAC nomenclature. Rhamnetin-3-O-rhamnoside-4′-O-(6″-O-cinnamoyl)-glucoside is one of the major indicator components of Loranthus tanakae Franch. & Sav, and it is a novel component that has not been reported in the academic world to date. The inventors of the present invention named it “Lotanoside B.”

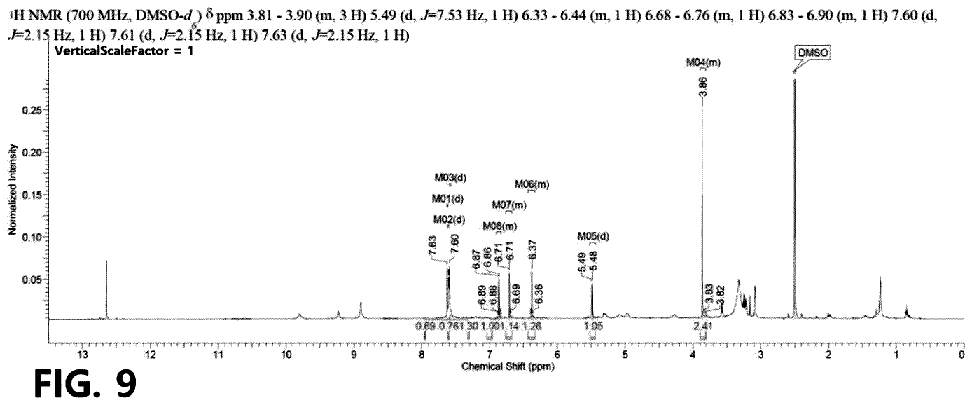

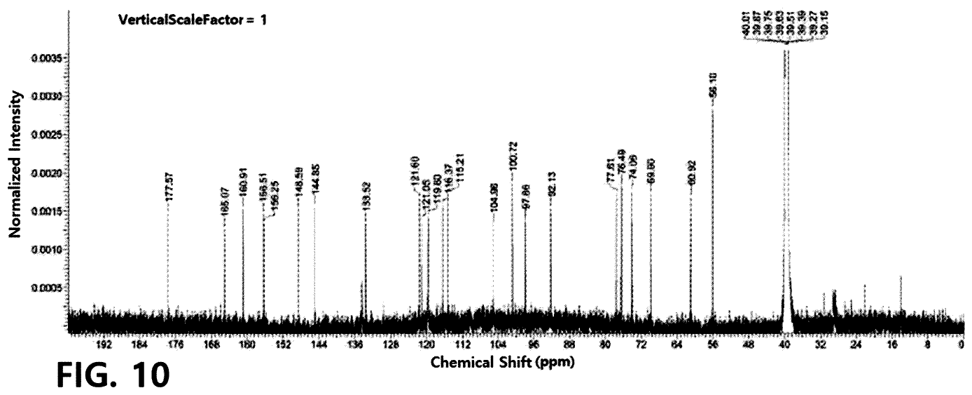

In addition, the compound structure of the component indicated as 2 in the HPLC pattern of the 70% ethanol extract of FIG. 2 was determined through 1D-NMR analysis. As shown in FIGS. 9 and 10, the compound of the component indicated as 2 was a known compound designated as rhamnetin-3-O-glucoside according to the IUPAC nomenclature, but it is a major indicator component that has been isolated and confirmed for the first time in Loranthus tanakae Franch. & Sav.

In the present invention, the isomer may include a racemate, an enantiomer, a diastereomer a mixture of enantiomers, or a mixture of diastereomers, but is not limited thereto.

In the present invention, the Lotanoside B may be isolated from Loranthus tanakae Franch. & Sav. Preferably, the Lotanoside B may be isolated from the root, flower, stem leaf, fruit or whole plant of Loranthus tanakae Franch. & Sav, which is a hemiparasite to Quercus and Castanea in the Fagaceae family, may be purchased commercially or may be collected or cultivated in nature.

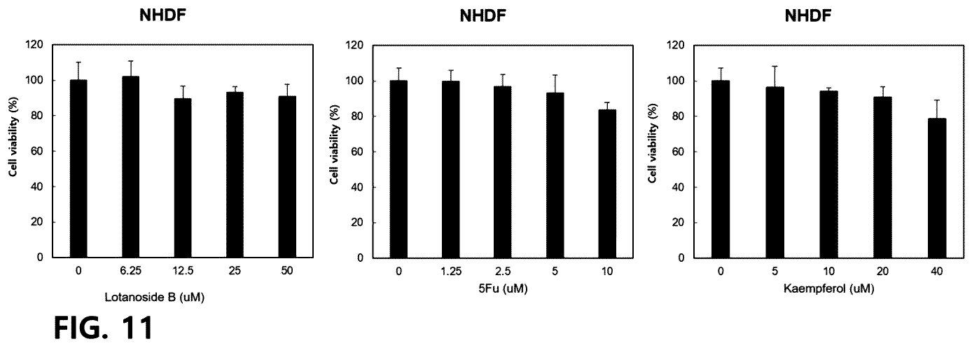

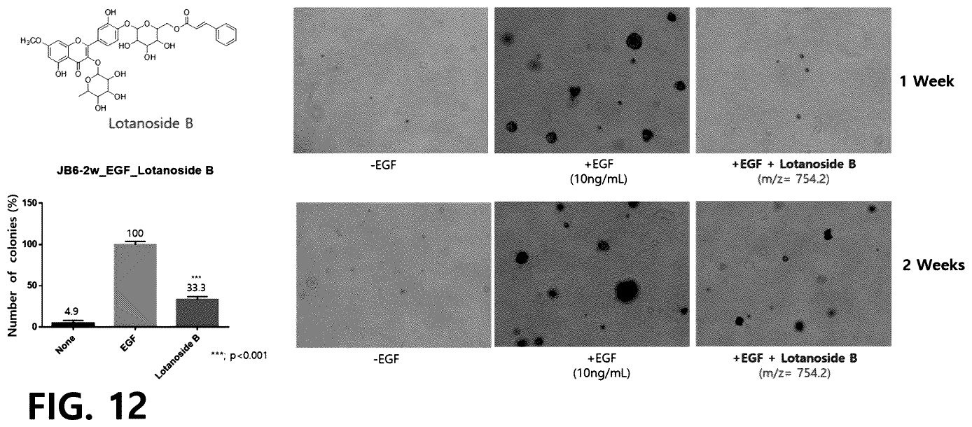

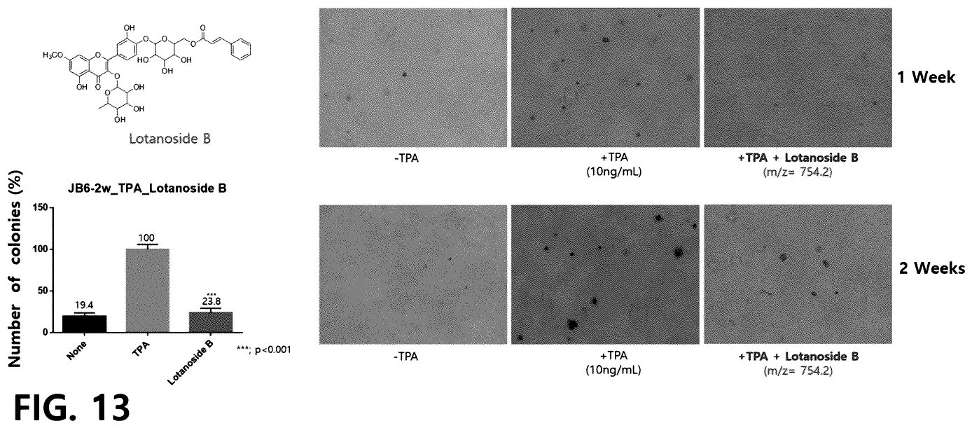

In a specific embodiment of the present invention, the toxicity and anticancer activity of individual components of Lotanoside B were evaluated. As a result, as confirmed in FIG. 11, Lotanoside B showed no toxicity up to 50 μM in normal human skin fibroblasts, confirming that it does not show toxicity even at relatively high concentrations compared to the existing anticancer agent 5-FU and kaempferol known to exhibit anticancer activity. In addition, as confirmed in FIGS. 12 and 13, Lotanoside B showed an inhibition rate of 66.7% compared to the EGF treatment group in the EGF-induced carcinogenesis model, and a 76.2% inhibition rate compared to the TPA treatment group in the TPA-induced carcinogenesis model, indicating that it exhibits an excellent tumor development inhibition effect or tumor development delay effect.

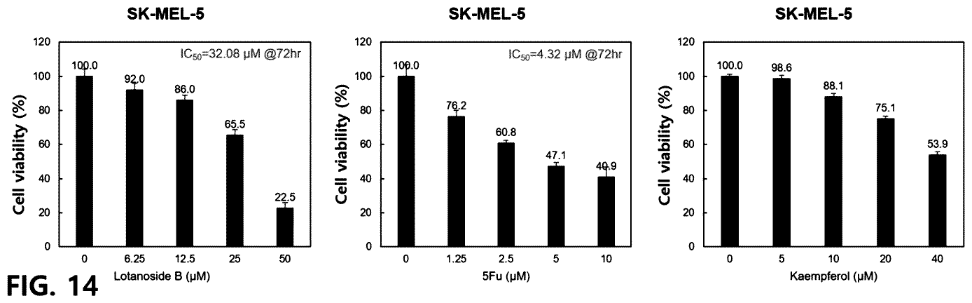

In another specific embodiment of the present invention, in order to evaluate the cancer cell inhibition activity of Lotanoside B, a WST-8 assay was performed by using SKMEL-5 melanoma cancer cells. As confirmed in FIG. 14, Lotanoside B inhibited the growth of cancer cells in a concentration-dependent manner, and the IC₅₀ value was measured as 32.08 μM. In addition, Lotanoside B not only exhibited an excellent cancer cell growth inhibition effect at the same concentration compared to kaempferol, which is known to exhibit anticancer activity, but also, unlike 5-FU, exhibited a cancer cell growth inhibition effect of 78.5% at a concentration that did not exhibit toxicity, and thus, it was confirmed that it exhibits an excellent anticancer effect without toxicity.

Accordingly, a second aspect of the present invention relates to a pharmaceutical composition including Lotanoside B represented by Chemical Formula 1 above, an isomer thereof or a pharmaceutically acceptable salt as an active ingredient, and a health functional food composition including Lotanoside B represented by Chemical Formula 1 above, an isomer thereof or a sitologically acceptable salt as an active ingredient.

In the pharmaceutical and health functional food compositions of the present invention, the compound, isomer thereof, pharmaceutically acceptable salt thereof or sitologically acceptable salt may be used for the purpose of preventing, ameliorating or treating cancer.

The cancer may be, for example, melanoma, non-melanoma skin cancer (e.g., squamous cell carcinoma), breast cancer, head and neck cancer, thyroid cancer, fibrosarcoma, soft tissue sarcoma, osteosarcoma, testicular cancer, prostate cancer, ovarian cancer, bladder cancer, skin cancer, brain cancer, hemangiosarcoma, mast cell tumor, leukemia, lymphoma, liver cancer, lung cancer, pancreatic cancer, stomach cancer, kidney cancer, colon cancer, hematopoietic tumor, neuroblastoma, epidermoid carcinoma or metastatic cancer thereof, but is not limited thereto.

With regard to the second aspect, the present invention further relates to a method for preventing, ameliorating or treating cancer, including administering Lotanoside B represented by Chemical Formula 1 above, an isomer thereof or a pharmaceutically acceptable salt thereof to a subject in need thereof.

With regard to the second aspect, the present invention further relates to the use of Lotanoside B represented by Chemical Formula 1 above, an isomer thereof or a pharmaceutically or sitologically acceptable salt thereof in the manufacture of a medicament or health functional food for preventing, ameliorating or treating cancer.

In the method and use of the present invention, the description of Lotanoside B represented by Chemical Formula 1 above, an isomer thereof or a pharmaceutically or sitologically acceptable salt thereof and the effects thereof are the same as described above, and therefore, the description thereof is omitted.

A third aspect of the present invention relates to a pharmaceutical composition for preventing or treating cancer and a health functional food composition for preventing or ameliorating cancer, which includes an extract of Loranthus tanakae Franch. & Sav including Lotanoside B represented by Chemical Formula 1 above or a fraction thereof as an active ingredient.

Since the above-mentioned Lotanoside B is isolated from Loranthus tanakae Franch. & Sav, the extract of Loranthus tanakae Franch. & Sav including the same or a fraction thereof may also be utilized for preventing, ameliorating or treating cancer.

The term “extract” used in the present invention includes an extract obtained by extracting the Loranthus tanakae Franch. & Sav, a diluted or concentrated solution of the extract, a dried product obtained by drying the extract, a controlled or purified product of the extract, or a mixture thereof, and all formulations that can be formed by using the extract. The extract may be extracted from a natural, hybrid or mutant plant of Loranthus tanakae Franch. & Sav, and may also be extracted from a plant tissue culture

The extract may be prepared by using hexane, chloroform, methylene chloride, ethyl acetate, acetone, a C₁ to C₄ lower alcohol, water or mixed solvent thereof as an extraction solvent, and preferably, it may be prepared by using a C₁ to C₄ lower alcohol, water or mixed solvent thereof as an extraction solvent, but is not limited thereto.

In the composition of the present invention, the extract of Loranthus tanakae Franch. & Sav may be prepared by extracting a whole Loranthus tanakae Franch. & Sav plant at least once using the extraction solvent, and may be prepared as a dried extract obtained by concentrating the solvent extract under reduced pressure and then freeze-drying or spray-drying the same.

The term “fraction” used in the present invention means a result obtained by performing fractionation to isolate a specific component or a specific component group from a mixture including various components.

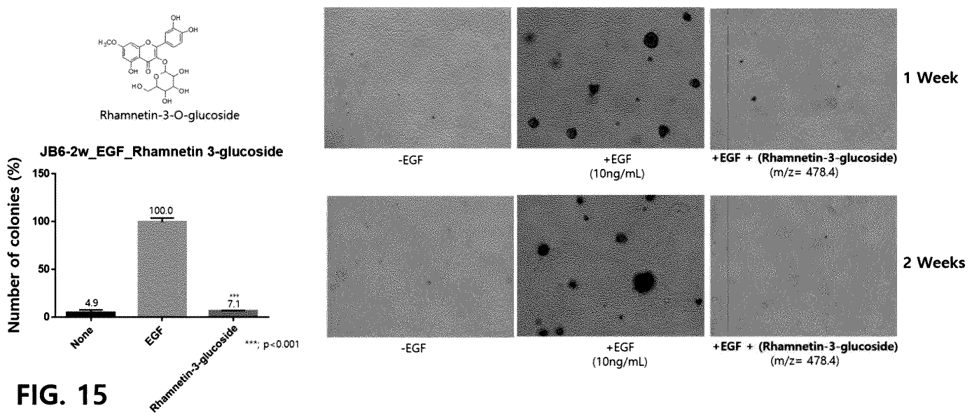

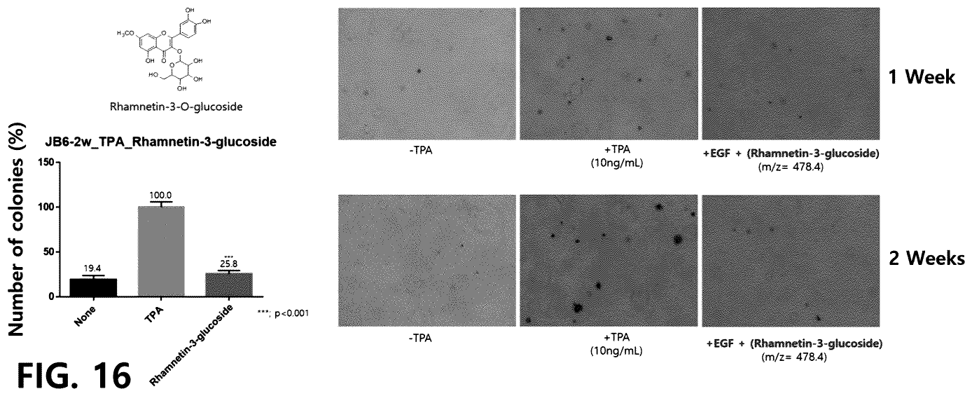

As described above, the inventors of the present invention confirmed that the extract of Loranthus tanakae Franch. & Sav includes not only Lotanoside B but also rhamnetin-3-O-glucoside. Although rhamnetin-3-O-glucoside is a known compound, it is the main component that was first isolated and confirmed from Loranthus tanakae Franch. & Sav, and the anticancer efficacy is unknown. Accordingly, in a specific embodiment of the present invention, the anticancer activity of rhamnetin-3-O-glucoside was evaluated in an EGF-induced carcinogenesis model and a TPA-induced carcinogenesis model. As a result, as confirmed in FIGS. 15 and 16, rhamnetin-3-O-glucoside showed an inhibition rate of 92.9% compared to the EGF treatment group in the EGF-induced carcinogenesis model, and an inhibition rate of 74.2% compared to the TPA treatment group in the TPA-induced carcinogenesis model, indicating that rhamnetin-3-O-glucoside also showed an excellent tumor development inhibition effect or tumor development delay effect.

Therefore, the extract of Loranthus tanakae Franch. & Sav or fraction thereof included in the composition of the present invention may be manufactured in a form including Lotanoside B and/or rhamnetin-3-O-glucoside.

In the composition of the present invention, the extract of Loranthus tanakae Franch. & Sav including at least one of the two compounds may be a C₁ to C₄ lower alcohol, water or mixed solvent extract thereof. The C₁ to C₄ lower alcohol may be methanol, ethanol, propanol, isopropanol or butanol, but is not limited thereto. For example, a preferred example of a C₁ to C₄ lower alcohol may be ethanol. The mixed solvent of the C₁ to C₄ lower alcohol and water may be preferably 30 to 80% ethanol, but is not limited thereto.

In the composition of the present invention, the fraction of Loranthus tanakae Franch. & Sav may be an ethyl acetate fraction of a C₁ to C₄ lower alcohol, water or mixed solvent extract thereof. In this case, the ethyl acetate fraction of the mixed solvent extract of a C₁ to C₄ lower alcohol and water may be preferably an ethyl acetate fraction of a 30 to 80% ethanol extract.

For example, the extract of Loranthus tanakae Franch. & Sav including at least one of the above two compounds may be prepared by grinding a whole plant of Loranthus tanakae Franch. & Sav and then repeatedly extracting the same once or more times for 0.5 to 5 hours by using water or a 70% ethanol solvent as an extraction solvent, but is not limited thereto.

In addition, the fraction of the extract of Loranthus tanakae Franch. & Sav including at least one of the above two compounds may be prepared by suspending the water or 70% ethanol extract in water and then sequentially fractionating the same with n-hexane, methylene chloride, ethyl acetate and n-butanol to obtain an ethyl acetate fraction, but is not limited thereto.

In the composition of the present invention, the term “prevention” means any act of inhibiting or delaying the onset of a disease or condition. In the present invention, it means delaying the onset of cancer or inhibiting the onset.

In the composition of the present invention, the term “amelioration” means any act of improving or beneficially changing the state of a disease or condition, and in the present invention, it means ameliorating the symptoms of cancer.

In the composition of the present invention, the term “treatment” means any act of delaying, stopping or reversing the progression of a disease or condition, and in the present invention, it means relieving, alleviating, eliminating or reversing the symptoms of cancer.

With regard to the third aspect, the present invention further relates to a method for preventing, ameliorating or treating cancer, including administering an extract of Loranthus tanakae Franch. & Sav including Lotanoside B represented by Chemical Formula 1 above or a fraction thereof to a subject in need thereof.

With regard to the third aspect, the present invention further relates to the use of an extract of Loranthus tanakae Franch. & Sav including Lotanoside B represented by Chemical Formula 1 above or a fraction thereof in the manufacture of a medicament or health functional food for preventing, ameliorating or treating cancer.

In the method and use of the present invention, the extract of Loranthus tanakae Franch. & Sav or a fraction, and the description of their effects are the same as described above, and therefore, the description thereof is omitted.

The term “pharmaceutically acceptable salt” used in the present invention means any organic or inorganic addition salt of each of the compounds of Chemical Formulas 1 and 2 at a concentration that has a relatively non-toxic and harmless effective effect for a patient, and the side effects caused by the salt do not reduce the beneficial effects of the compounds of Chemical Formulas 1 and 2. These salts may use inorganic acids and organic acids as free acids, and inorganic acids such as hydrochloric acid, hydrobromic acid, nitric acid, sulfuric acid, perchloric acid, phosphoric acid and the like may be used, and organic acids such as citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid or malonic acid may be used. In addition, these salts include alkali metal salts (sodium salts, potassium salts, etc.) and alkaline earth metal salts (calcium salts, magnesium salts, etc.). For example, acid addition salts may include acetate, aspartate, benzate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthylate, 2-naphthylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts and the like, and among these, it may be hydrochloride or trifluoroacetate.

The pharmaceutical composition of the present invention may further include a pharmaceutically acceptable carrier.

The pharmaceutical composition of the present invention may be prepared into a pharmaceutical formulation using a method well known in the art so as to provide a rapid, sustained or delayed release of the active ingredient after administration to a mammal. In terms of preparing the formulation, the active ingredient is preferably mixed or diluted with a carrier, or enclosed in a carrier in the form of a container.

Therefore, the pharmaceutical composition of the present invention may be formulated and used in the form of oral formulations such as powders, granules, tablets, capsules, suspensions, emulsions, syrups, aerosols, external preparations, suppositories and sterile injection solutions according to conventional methods, and may further include appropriate carriers, excipients and diluents that are conventionally used in the preparation of compositions.

For example, carriers that can be included in the pharmaceutical composition of the present invention include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil. When it is formulated, it is prepared by using commonly used diluents or excipients such as fillers, bulking agents, binders, wetting agents, disintegrating agents and surfactants.

Solid preparations for oral administration include tablets, pills, powders, granules and capsules, and these solid preparations are prepared by mixing the compound with at least one excipient, such as starch, calcium carbonate, sucrose or lactose, gelatin and the like. In addition to simple excipients, lubricants such as magnesium stearate and talc are also used.

Liquid preparations for oral administration include suspensions, oral solutions, emulsions and syrups, and in addition to commonly used simple diluents such as water and liquid paraffin, various excipients such as wetting agents, sweeteners, fragrances and preservatives may be included.

Preparations for parenteral administration include sterile aqueous solutions, non-aqueous solvents, suspensions, emulsions, lyophilized preparations and suppositories. Non-aqueous solvents and suspensions may include propylene glycol, polyethylene glycol, vegetable oils such as olive oil, and injectable esters such as ethyl oleate. Bases of suppositories may include witepsol, macrogol, Tween 61, cacao butter, laurin butter, glycerogelatin and the like.

The term “administration” used in the present invention means introducing the pharmaceutical composition of the present invention to a patient by any appropriate method.

The method of administration of the pharmaceutical composition according to the present invention is not particularly limited and may follow a method commonly used in the art. The method of administration is not limited as long as it can reach the target tissue, but may include intraperitoneal administration, intravenous administration, intramuscular administration, subcutaneous administration, intradermal administration, oral administration, topical administration and intranasal administration. The pharmaceutical composition according to the present invention may be prepared in various dosage forms according to the intended administration method.

The pharmaceutical composition according to the present invention may be administered in a pharmaceutically effective amount.

The “pharmaceutically effective amount” means an amount that is sufficient to treat a disease with a reasonable benefit/risk ratio applicable to medical treatment, and the effective dosage level may be determined according to factors including the type and severity of the individual, age, gender, type of infected virus, activity of the drug, sensitivity to the drug, time of administration, route of administration and excretion rate, treatment period, concurrently used drugs, and other factors well known in the medical field.

The typical daily dosage of the pharmaceutical composition according to the present invention may be appropriately selected by a person skilled in the art, and it may be administered once or in several divided doses.

The composition of the present invention may be administered daily or intermittently, and the number of times of administration per day may be divided into one or two to three times. In addition, the composition of the present invention may be used alone or in combination with other drug treatments for the prevention or treatment of cancer. Considering all of the above factors, it is important to administer an amount that can achieve the maximum effect with the minimum amount without side effects, and this can be easily determined by a person skilled in the art.

The term “subject” used in the present invention means all animals, including humans, that have developed or are likely to develop cancer. In addition to humans, the animal may be a mammal such as a cow, horse, sheep, pig, goat, camel, antelope, dog or cat that requires treatment for symptoms similar to those of humans, but is not limited thereto.

The term “food” used in the present invention includes meat, sausage, bread, chocolate, candy, snacks, confectionery, pizza, ramen, other noodles, gum, dairy products including ice cream, various soups, beverages, tea, drinks, alcoholic beverages, vitamin complexes, health functional foods and health foods, and includes all foods in the conventional sense.

The health functional food composition of the present invention includes forms such as pills, powders, granules, infusions, tablets, capsules or liquids, and foods to which the composition of the present invention may be added include, for example, various foods, such as beverages, gums, tea, vitamin complexes, health supplements and the like.

There are no particular restrictions on other ingredients except for the essential ingredients that can be included in the health functional food composition of the present invention, such as Lotanoside B, rhamnetin-3-O-glucoside and an extract of Loranthus tanakae Franch. & Sav or a fraction thereof, and various herbal extracts, food supplement additives or natural carbohydrates may be included as additional ingredients, just like conventional foods.

In addition, the food auxiliary additive includes food auxiliary additives that are conventional in the art, such as flavoring agents, flavoring agents, coloring agents, fillers, stabilizers and the like.

Examples of the natural carbohydrates include monosaccharides, such as glucose, fructose and the like; disaccharides, such as maltose, sucrose and the like; and polysaccharides, such as dextrin, cyclodextrin and the like, common sugars, and sugar alcohols, such as xylitol, sorbitol, erythritol and the like. In addition to those described above, natural flavoring agents (such as rebaudioside A, glycyrrhizin, etc.) and synthetic flavoring agents (saccharin, aspartame, etc.) may be advantageously used as flavoring agents.

In addition to the above, the health functional food composition of the present invention may contain various nutrients, vitamins, minerals (electrolytes), flavoring agents such as synthetic flavoring agents and natural flavoring agents, coloring agents and fillers (cheese, chocolate, etc.), pectic acid and its salts, alginic acid and its salts, organic acids, protective colloid thickeners, pH regulators, stabilizers, preservatives, glycerin, alcohol, carbonating agents used in carbonated beverages and the like. In addition, it may contain fruit pulp for the production of natural fruit juice and fruit juice drinks and vegetable drinks. These ingredients may be used independently or in combination.

In the present invention, the health supplement includes health functional foods and health foods and the like.

The health functional food is the same term as food for special health use (FoSHU), and means a food with high medical and health effects that is processed to efficiently exhibit a bioregulatory function in addition to providing nutrition. Herein, “function (functional)” means regulating nutrients for the structure and function of the human body or obtaining a useful effect for health purposes such as physiological functions. The food of the present invention may be manufactured by a method commonly used in the art, and may be manufactured by adding raw materials and ingredients commonly added in the art during the manufacturing process. In addition, the formulation of the food may be manufactured without limitation as long as it is a formulation recognized as food. The health functional food composition of the present invention may be manufactured in various forms of formulations, and unlike general drugs, it has the advantage of not having side effects that may occur when taking drugs for a long period of time by using food as a raw material.

In addition, the composition of the present invention may be provided as an anticancer adjuvant.

The term “anticancer adjuvant” used in the present invention refers to something that is used to enhance the anticancer effect of an anticancer agent and suppress or improve the side effects of an anticancer agent, and it may be administered to a patient in combination with an anticancer agent.

In the present invention, the term “subject” includes, but is not limited to, any animal (e.g., human, horse, pig, rabbit, dog, sheep, goat, non-human primate, cow, cat, guinea pig or rodent). This term does not indicate a specific age or gender. Therefore, it is intended to include adult/adult and newborn subjects, as well as fetuses, whether female/female or male/male. A patient refers to a subject suffering from a disease or disorder. The term patient includes human and veterinary subjects.

In the method of the present invention, the effects of Lotanoside B represented by Chemical Formula 1 above, an isomer thereof, a pharmaceutically or sitologically acceptable salt thereof, or the constitutions including the route of administration, the number of times of administration and the dosage of an extract of Loranthus tanakae Franch. & Sav according to the present invention or a fraction are the same as described above, and thus, the description thereof is omitted.

A fourth aspect of the present invention relates to a method for isolating Lotanoside B represented by Chemical Formula 1 above and/or rhamnetin-3-O-glucoside represented by Chemical Formula 2 from Loranthus tanakae Franch. & Sav, including the following steps:
(a) extracting Loranthus tanakae Franch. & Sav with an ethanol solvent to obtain a C₁ to C₄ lower alcohol, water or mixed solvent extract thereof;
(b) sequentially fractionating the extract with n-hexane, methylene chloride, ethyl acetate and n-butanol in this order to obtain an ethyl acetate fraction;
(c) performing flash chromatography on the ethyl acetate fraction using a water-methanol mixture solution as an extract solvent to obtain a fraction;
(d) performing reverse-phase flash chromatography on the fraction obtained after performing flash chromatography using a methanol solvent as an extract solvent to obtain a fraction; and
(e) performing reverse-phase flash chromatography and then performing normal-phase flash chromatography on the fraction obtained using a chloroform-methanol-water mixture solution as an extraction solvent to purify Lotanoside B represented by Chemical Formula 1 above and/or rhamnetin-3-O-glucoside represented by Chemical Formula 2 above.

In the method of the present invention, the Loranthus tanakae Franch. & Sav of step (a) may be dried through a conventional drying method such as natural drying or hot air drying.

In the method of the present invention, the type of extraction solvent used to extract Loranthus tanakae Franch. & Sav in step (a) may be a C₁ to C₄ lower alcohol, water or mixed solvent thereof. The C₁ to C₄ lower alcohol may be methanol, ethanol, propanol, isopropanol or butanol, but is not limited thereto. For example, a preferred example of a lower alcohol of C₁ to C₄ may be ethanol. Extraction using ethanol (i.e., alcohol extraction) is the most environmentally friendly and economical extraction method in food processing. The ethanol may be, for example, 30% to 80% ethanol, but is not limited thereto.

The method for preparing the extract is not particularly limited, and may be extracted according to a method commonly used in the relevant technical field. Non-limiting examples of the extraction method include immersion extraction, hot water extraction, ultrasonic extraction, filtration, reflux extraction and the like, and these may be performed alone or in combination of two or more methods.

In the method of the present invention, step (b) may be performed after suspending the dried form of the extract of Loranthus tanakae Franch. & Sav obtained in step (a) in water.

In the method of the present invention, the water-methanol mixture of step (c) may be applied with a concentration gradient from 100% water to 100% methanol, and the flash chromatography may be performed by using a column filled with a standard reversed-phase hydrophobic resin, for example, a C18 resin.

In the method of the present invention, the methanol solvent of step (d) may be applied with a concentration gradient from 50% methanol to 100% methanol, and the reversed-phase flash chromatography may be performed by using a column filled with a standard reversed-phase hydrophobic resin, for example, a C18 resin.

In the method of the present invention, the chloroform-methanol-water mixture solution of step (e) may be applied with a concentration gradient of 7 to 9:1 to 3:0.1 to 2, and the flash chromatography may be performed as a semi-preparative chromatography using a column filled with a standard normal phase polar resin, for example, silica gel.

Hereinafter, the present invention will be described in more detail by the following examples. However, the following examples are only for illustrating the present invention, and the scope of the present invention is not limited thereto.

### MODES OF THE INVENTION

### Example 1

Preparation of Extract of Loranthus tanakae Franch. & Sav and Confirmation of its Anticancer Effect
1-1. Preparation of Extract of Loranthus tanakae Franch. & Sav

1.3 kg of Loranthus tanakae Franch. & Sav (Jeongseon, Gangwon-do) was crushed and extracted twice with 6 L of 70% ethanol for 2 hours each. The extract was filtered through a filter paper, the filtrate was collected, and the filtrate was concentrated in a concentrator to obtain 126.42 g of the 70% ethanol extract of Loranthus tanakae Franch. & Sav (yield 12.25%), which was then stored at −20° C.

1-2. Confirmation of the Anticancer Effect of the Extract of Loranthus tanakae Franch. & Sav

The carcinogenicity inhibitory effect of the extract of Loranthus tanakae Franch. & Sav was determined in a carcinogenic model in which the mouse skin epithelial cell JB6 (ATCC, USA) was treated with EGF, which is a growth factor that increases cancer cell occurrence, and TPA, which is a tumor-promoting factor, to induce malignant transformation.

Specifically, agar mix (0.5%) including BME, FBS, glutamine, gentamicin, PBS and 1.25% agar, EGF (10 ng/ml) or TPA (10 ng/ml) and the 70% ethanol extract of Loranthus tanakae Franch. & Sav prepared in Example 1-1 were mixed, dispensed into 6-well plates at 3 mL per well, and solidified (bottom agar). Mouse skin epithelial cells JB6 (ATCC, USA) were detached by trypsin treatment, washed with 1×PBS, and resuspended in 10% BME to determine the cell number. 8,000 cells were prepared per well, and agar mix (0.3%), EGF or TPA and the 70% ethanol extract of Loranthus tanakae Franch. & Sav prepared in Example 1-1 were mixed, dispensed into 1 mL per well, and solidified (upper agar). After culturing in a CO₂ incubator for about 2 weeks, colonies were photographed under a microscope, and the number of colonies was counted by using Image-Pro Plus software (v.6.0) program (Media Cybernetics, Silver Spring, MD, USA).

As a result, as confirmed in FIG. 1, the test group treated with the 70% ethanol extract of Loranthus tanakae Franch. & Sav showed an inhibition rate of 42.5% compared to the EGF treatment group in the EGF-induced carcinogenesis model, and a 65.9% inhibition rate compared to the TPA treatment group in the TPA-induced carcinogenesis model. Through this, it was confirmed that the 70% ethanol extract of Loranthus tanakae Franch. & Sav has an effect of inhibiting or delaying tumor development.

### Example 2

Isolation of Compounds Derived from Extract of Loranthus tanakae Franch. & Sav
2-1. Identification of Individual Components of Extract of Loranthus tanakae Franch. & Sav

The 70% ethanol extract of Loranthus tanakae Franch. & Sav was dissolved in 70% ethanol to prepare a sample for HPLC analysis, and the sample was filtered immediately before analysis and pattern analysis was performed. The equipment used for the analysis was an HPLC from Waters Co., and a photodiode array detector and a quadrupole mass spectrometer were used as component detectors. The analysis wavelength was 200 to 400 nm, the components were detected at 254 nm, and the molecular weight range was scanned in the negative mode for 200 to 1,000 m/z values. The column used was Luna C18(2) 100A (4.6× 250 mm, 5 μm), and 10 μL of the sample was injected for analysis. For the solvent, water including 0.1% formic acid and acetonitrile including 0.1% formic acid were used to change the gradient from 95% water to 100% acetonitrile for 45 minutes. As a result of the analysis, as confirmed in FIG. 2, the compound indicated as 1 was detected as a component with a molecular weight of 753.22 m/z at a retention time of about 36.8 minutes, and the compound indicated as 2 was detected as a component with a molecular weight of 477.18 m/z at a retention time of about 19.9 minutes.

2-2. Isolation of Compounds Derived from Loranthus tanakae Franch. & Sav

38.63 g of the 70% ethanol extract prepared in Example 1-1 was suspended in 700 mL of water and fractionated sequentially with n-hexane, methylene chloride, ethyl acetate and n-butanol to obtain 8.6 g (yield 22.26%) of ethyl acetate (EA).

The EA fraction was separated from 100% water to 100% methanol using a flash chromatography system (Biotage, Sweden) and a column filled with Diaion HP-20 resin, and a total of 10 fractions (F1 to F10) were obtained after TLC confirmation. Among these, the F9 fraction was eluted from 50% methanol to 70% methanol with sfar C18 (400 g, Biotage), confirmed by TLC, and divided again into 11 subfractions (F9-01 to F9-11). Among these, the F9-10 fraction was eluted from chloroform:methanol:DW=9:1:0.1 to 8:2:0.2 using a sfar silica HC (25 g, Biotage) cartridge to isolate a novel compound. The isolated component was a light yellow powder, and as a result of determining the structure by HPLC, HRMS, 1D-NMR and 2D-NMR, it was confirmed to be a novel compound designated as rhamnetin-3-O-rhamnoside-4′-O-(6″-O-cinnamoyl)-glucoside according to the IUPAC nomenclature as shown in FIGS. 2 to 8, and it was named Lotanoside B.

In addition, the EA fraction was separated from 100% water and 100% methanol using a flash column chromatography system (Biotage, Sweden) and a column filled with Diaion HP-20 resin, and after confirmation by TLC, a total of 10 fractions (F1 to F10) were obtained. Among these, the F7 fraction was eluted from 30% methanol to 70% methanol with sfar C18 (400 g, Biotage), and after confirmation by TLC, it was separated again into 8 subfractions (F7-01 to F7-08). Rhamnetin-3-O-glucoside was isolated from the F7-2 fraction by eluting the same from chloroform:methanol:DW=9:1:0.1 to 8:2:0.2 using a sfar silica HC (25 g, Biotage) cartridge. The isolated component was dark yellow, and as a result of determining the structure by referring to HPLC, MS 1D-NMR and existing literature, it was confirmed to be a compound designated as rhamnetin-3-O-glucoside according to the IUPAC nomenclature, as shown in FIGS. 2, 9 and 10. Rhamnetin-3-O-glucoside is a known compound, but it was first isolated from Loranthus tanakae Franch. & Sav.

### Example 3

Confirmation of Toxicity and Anticancer Effect of Novel Compound Derived from Loranthus tanakae Franch. & Sav 3-1. Toxicity test in normal cells

NHDF (normal human dermal fibroblast; ATCC, USA) cells were seeded at about 5,000 cells per well in a 96-well plate and stabilized for 24 hours, and then treated with Lotanoside B isolated in Example 2 at various concentrations (0, 6.25, 12.5, 25, or 50 μM). As a control group, 5-FU (anticancer drug) and kaempferol were treated at various concentrations (5-FU: 0, 1.25, 2.5, 5 or 10 μM; kaempferol: 0, 5, 10, 20 or 40 μM). After culturing in a CO₂ incubator for 48 hours, WST-8 ((2-(2-methoxy-4-nitrophenyl)-3-(4-nitrophenyl)-5-(2,4-disulfophenyl)-2H-tetrazolium, monosodium salt) reagent was treated, and after culturing for 2 hours, the optical density (OD) was measured at a wavelength of 450 nm by using a microplate reader.

As confirmed in FIG. 11, 5-FU began to show toxicity at 10 μM and kaempferol at 40 μM, whereas the cytotoxicity of Lotanoside B was not observed up to 50 μM, confirming that no toxicity was observed even at high concentrations.

### 3-2. Confirmation of Anticancer Effect

The cancer-inducing inhibitory effect of Lotanoside B was determined by using the same method as in Example 1-2. In this case, Lotanoside B was treated at a concentration of 50 μM.

As confirmed in FIG. 12, the test group treated with Lotanoside B showed a 66.7% inhibition rate compared to the EGF treatment group in the EGF-induced carcinogenesis model, and as confirmed in FIG. 13, it showed a 74.2% inhibition rate compared to the TPA treatment group in the TPA-induced carcinogenesis model. Through this, it was confirmed that Lotanoside B, which is a novel compound isolated from Loranthus tanakae Franch. & Sav, showed an excellent tumor development inhibition or tumor development delay effect.

### 3-3. Confirmation of Cancer Cell Growth Inhibition Effect

SKMEL-5 melanoma cancer cells (ATCC, USA) were dispensed into a 96-well plate at 1,000 cells per well and stabilized for 24 hours, and then treated with Lotanoside B at different concentrations (0, 6.25, 12.5, 25 or 50 μM). As a control group, 5-FU (anticancer drug) and kaempferol were treated at different concentrations, respectively (5-FU: 0, 1.25, 2.5, 5 or 10 μM; kaempferol: 0, 5, 10, 20 or 40 μM). Afterwards, the cells were cultured in a CO₂ incubator for 72 hours, and then treated with WST-8 ((2-(2-methoxy-4-nitrophenyl)-3-(4-nitrophenyl)-5-(2,4-disulfophenyl)-2H-tetrazolium, monosodium salt) reagent, and after culturing for about 2 hours, the optical density (OD) was measured at a wavelength of 450 nm by using a microplate reader.

As shown in FIG. 14, the IC₅₀ value of Lotanoside B was confirmed to be 32.08 μM, and it was confirmed to exhibit a superior cancer cell growth inhibition effect compared to kaempferol.

### Example 4

Confirmation of Anticancer Effects of Other Compounds Derived from Loranthus tanakae Franch. & Sav

Rhamnetin-3-O-glucoside (CAS Number 27875-34-9) isolated in Example 2 is a major indicator component isolated for the first time from Loranthus tanakae Franch. & Sav, and its anticancer effects have not been reported. Therefore, in this example, the anticancer inhibitory effect of rhamnetin-3-O-glucoside was determined by using the same method as Example 1-2.

As confirmed in FIG. 15, the test group treated with rhamnetin-3-O-glucoside showed an inhibition rate of 92.9% compared to the EGF treatment group in the EGF-induced carcinogenesis model, and as confirmed in FIG. 16, it showed an inhibition rate of 76.2% in the TPA-induced carcinogenesis model. Through this, it was confirmed that rhamnetin-3-O-glucoside, which was isolated for the first time from Loranthus tanakae Franch. & Sav, also showed an excellent tumor occurrence inhibition or tumor occurrence delay effect.

### Example 5

### Confirmation of Anticancer Effect in Animal Model

Female ICR mice (5 weeks old, 20~25 g) were purchased as experimental animals from Samtaco Co., Ltd. (Osan, Korea), and were provided with water and food freely in a cage equipped with a constant temperature and humidity device. After a one-week adaptation period, the hair on the back of the mice was removed, and they were divided into six groups as shown in Table 1 and raised for 20 weeks.

TABLE 1

| Group | DMBA/TPA | Drug Treatment |
|---|---|---|
| Normal Group (Control Group) | — | Vehicle |
| Skin Cancer-Induced Group | DMBA/TPA | — |
| Skin Cancer-Induced and | | Lotanoside B (0.1 μmole) |
| Lotanoside B(C3)-Treated | | Lotanoside B (1 μmole) |
| Group | | |
| Skin Cancer-Induced and | | 5-FU(0.1 μmole) |
| 5-Fu-Treated Group | | |
| Lotanoside B(C3)-Treated | — | Lotanoside (1 μmole) |
| Group | | |

When DMBA (7,12-Dimethylbenz[a]anthracene), which induces carcinogenesis, is treated to the depilated area and TPA (12-Otetradecanoylphorbol-13-acetate, promoter) is continuously applied, skin inflammation and skin cancer are induced. In this case, as shown in Table 1, the treatment group treated with a low concentration (0.1 μmole) and a high concentration (1 μmole) of Lotanoside B and the positive control group treated with 5-FU were observed for skin carcinogenesis for 20 weeks. TPA, Lotanoside B and 5-FU were diluted in acetone and topically treated to the back area twice a week, and the number and size of the tumors formed and the weight of the mice were measured.

This animal experiment was conducted with the approval of the Dongshin University Animal Experiment Ethics Committee (Approval Number:DSU2022-03-03), and the handling and management of experimental animals followed the Animal Experiment Handling Regulations of the Experimental Animal Ethics Committee.

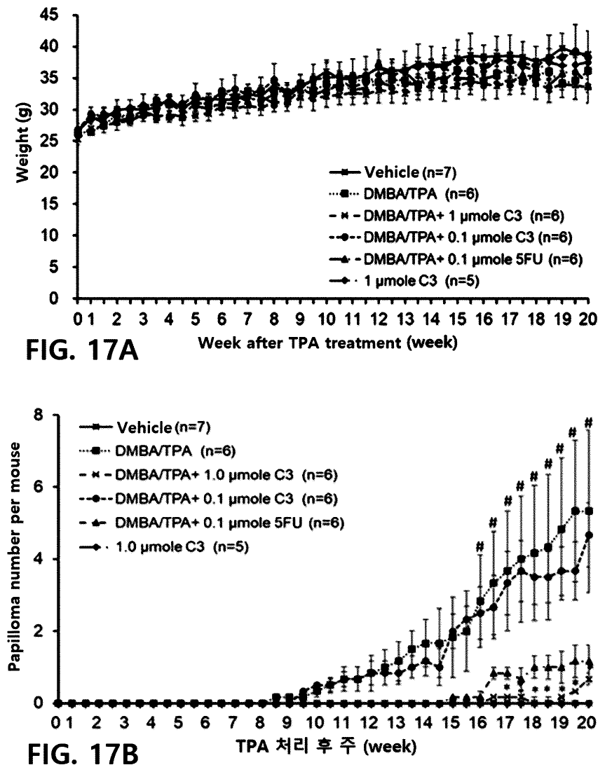

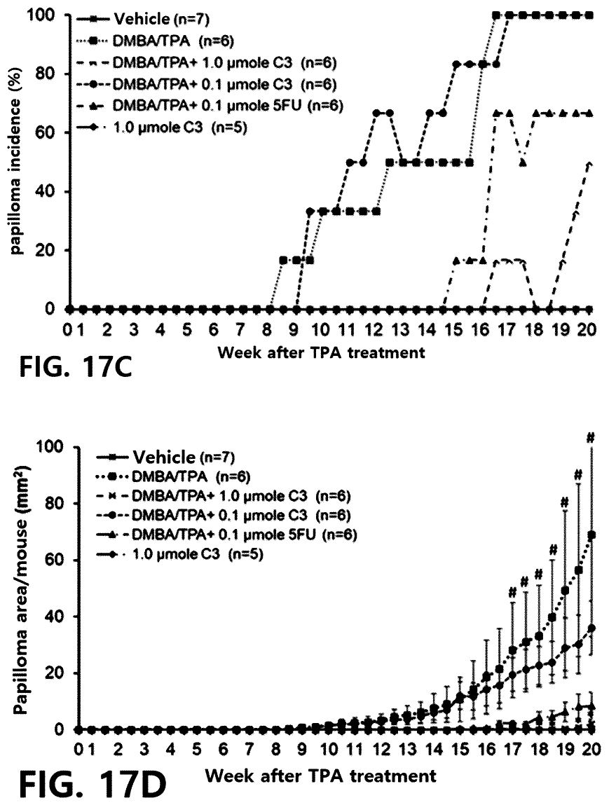

As shown in FIG. 17A, Lotanoside B did not affect the body weight of the mice. As shown in FIGS. 17B to 17D, the skin cancer-induced group treated with DMBA/TPA showed an increase in the number of tumors (FIG. 17B), tumor incidence (FIG. 17C) and tumor area (FIG. 17D) compared to the normal control group, but when Lotanoside B was treated together, the number of tumors (FIG. 17B), tumor incidence (FIG. 17C) and tumor area (FIG. 17D) decreased. This reduction effect was greater in the group treated with high concentrations of Lotanoside B (1 μmole), indicating that the skin cancer suppression effect increased in a dose-dependent manner of Lotanoside B. In addition, it was confirmed that Lotanoside B had a superior effect in inhibiting skin cancer formation compared to 5-FU used as a positive control.

From the above description, those skilled in the art will be able to understand that the present invention can be implemented in other specific forms without changing the technical idea or essential features thereof. In this regard, it should be understood that the embodiments described above are exemplary in all respects and not limiting. The scope of the present invention should be interpreted as including all changes or modifications derived from the meaning and scope of the patent claims described below rather than the detailed description above and the equivalent concepts thereof within the scope of the present invention.

The national research and development project that supported the present invention is as follows.
[Project Unique Number] 1711175244
[Project Number] KSN2021320
[Name of Ministry] Ministry of Science and ICT
[Name of Project Management (Specialized) Institution] Korea Institute of Oriental Medicine
[Title of Research Project] Korea Institute of Oriental Medicine Research Operation Expense Support (Main Project Expense)
[Title of Research Task] Development of Sustainable Traditional Korean Medicine Standard Resource Utilization Technology
[Contribution Ratio] 1/1
[Name of Project Performance Institution] Korea Institute of Oriental Medicine
[Research Period] Jan. 1, 2019 to Dec. 31, 2022

1. Lotanoside B represented by Chemical Formula 1, an isomer thereof or a pharmaceutically acceptable salt thereof:

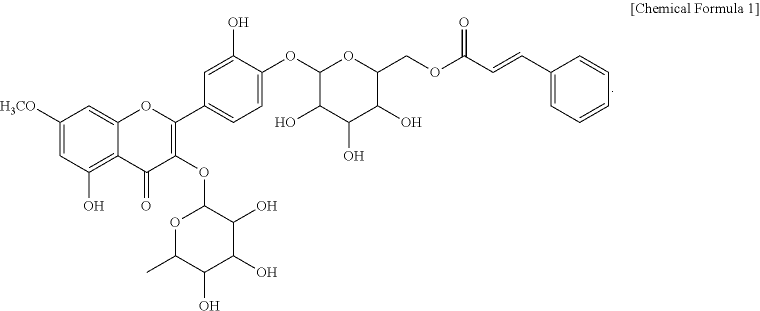 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1]

2. The compound, isomer thereof or pharmaceutically acceptable salt thereof according to claim 1, wherein the isomer comprises a racemate, an enantiomer, a diastereomer, a mixture of enantiomers or a mixture of diastereomers.

3. The compound, isomer thereof or pharmaceutically acceptable salt thereof according to claim 1, wherein the Lotanoside B is isolated from Loranthus tanakae Franch. & Sav.

4. The compound, isomer thereof or pharmaceutically acceptable salt thereof according to claim 3, wherein the Lotanoside B is isolated from a whole plant of Loranthus tanakae Franch. & Sav.

5. The compound, isomer thereof or pharmaceutically acceptable salt thereof according to claim 1, wherein the Lotanoside B, isomer thereof or pharmaceutically acceptable salt thereof has an effect of inhibiting cancer cells or inhibiting or delaying tumor development.

6. A pharmaceutical composition, comprising the compound, isomer thereof or pharmaceutically acceptable salt thereof according to claim 1 as an active ingredient.

7. The pharmaceutical composition of claim 6, wherein the compound, isomer thereof or pharmaceutically acceptable salt thereof is for preventing or treating cancer.

8. The pharmaceutical composition of claim 6, wherein the pharmaceutical composition is used as an anticancer adjuvant.

9. (canceled)

10. (canceled)

11. (canceled)

12. (canceled)

13. A health functional food composition, comprising the compound according to claim 1, an isomer thereof or a sitologically acceptable salt thereof as an active ingredient.

14. The health functional food composition of claim 13, wherein the compound, isomer thereof or sitologically acceptable salt thereof is for preventing or ameliorating cancer.

15. (canceled)

16. (canceled)

17. (canceled)

18. A method for isolating Lotanoside B represented by Chemical Formula 1 below and/or rhamnetin-3-O-glucoside represented by Chemical Formula 2 below from Loranthus tanakae Franch. & Sav, the method comprising: 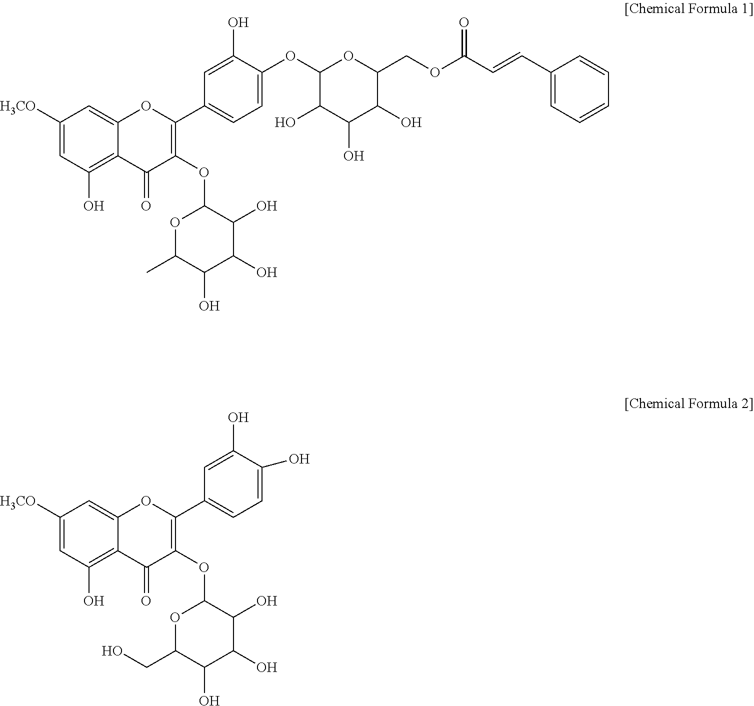 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1 ; COc1cc(O)c2c(=O)c(OC3OC(CO)C(O)C(O)C3O)c(-c3ccc(O)c(O)c3)oc2c1]
(a) extracting Loranthus tanakae Franch. & Sav with an ethanol solvent to obtain a C₁ to C₄ lower alcohol, water or mixed solvent extract thereof;
(b) sequentially fractionating the extract with n-hexane, methylene chloride, ethyl acetate and n-butanol in this order to obtain an ethyl acetate fraction;
(c) performing flash chromatography on the ethyl acetate fraction using a water-methanol mixture solution as an extract solvent to obtain a fraction;
(d) performing reverse-phase flash chromatography on the fraction obtained after performing flash chromatography using a methanol solvent as an extract solvent to obtain a fraction; and
(e) performing reverse-phase flash chromatography and then performing normal-phase flash chromatography on the fraction obtained using a chloroform-methanol-water mixture solution as an extraction solvent to purify Lotanoside B represented by Chemical Formula 1 above and/or rhamnetin-3-O-glucoside represented by Chemical Formula 2 above.

19. The method of claim 18, wherein the Loranthus tanakae Franch. & Sav of step (a) uses a whole plant.

20. The method of claim 18, wherein the mixed solvent of a C₁ to C₄ lower alcohol and water in step (a) is a 30 to 80% ethanol solvent.

21. The method of claim 18, wherein the water-methanol mixture solution of step (c) is applied with a concentration gradient from 100% water to 100% methanol.

22. The method of claim 18, wherein the methanol solvent of step (d) is applied with a concentration gradient from 50% methanol to 100% methanol.

23. The method of claim 18, wherein the chloroform-methanol-water mixture solution of step (e) is applied at a concentration gradient of 7 to 9:1 to 3:0.1 to 2.

24. A method for preventing, ameliorating or treating cancer, comprising administering the compound, isomer thereof or pharmaceutically acceptable salt thereof according to claim 1 to a subject in need thereof:

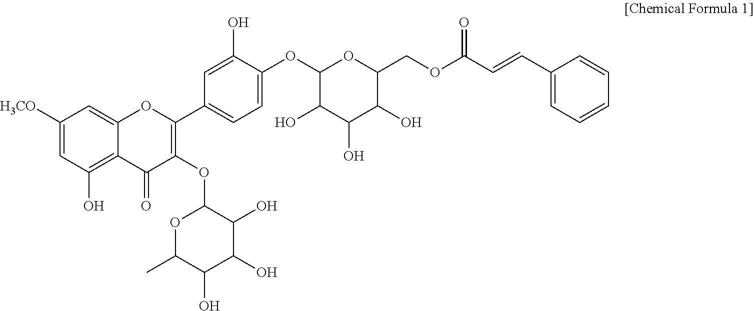 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1]

25. A method for preventing, ameliorating or treating cancer, comprising administering an extract of Loranthus tanakae Franch. & Sav comprising the compound according to claim 1 or a fraction thereof to a subject in need thereof:

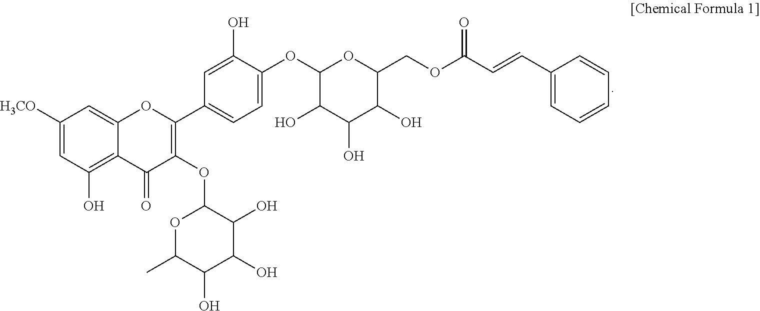 [SMILES: COc1cc(O)c2c(=O)c(OC3OC(C)C(O)C(O)C3O)c(-c3ccc(OC4OC(COC(=O)/C=C/c5ccccc5)C(O)C(O)C4O)c(O)c3)oc2c1]

26. (canceled)

27. (canceled)
